(19) United States
(12) Patent Application Publication
(10) Pub. No.: US 2024/0016687 A1
(43) Pub. Date: Jan. 18, 2024

(54) MODULAR ROBOTIC PATIENT AND MEDICAL TOOL POSITIONING SYSTEM

(71) Applicant: Spinotron Magyar Sugársebészeti Innováció Korlátolt Felelosségu Társaság, Debrecen (HU)

(72) Inventors: Xiao Ran Zheng, Central, SC (US); Donald Medlin, Greenville, SC (US); Jad Saud, Debrecen (HU)

(73) Assignee: Spinotron Magyar Sugársebészeti Innováció Korlátolt Felelosségu Társaság, Debrecen (HU)

(21) Appl. No.: 18/373,090

(22) Filed: Sep. 26, 2023

Related U.S. Application Data
(63) Continuation-in-part of application No. 17/271,030, filed on Feb. 24, 2021.
(60) Provisional application No. 62/722,506, filed on Aug. 24, 2018.
(60) Provisional application No. 62/722,488, filed on Aug. 24, 2018.

(51) Int. Cl. A61G 13/02; A61G 13/10; A61B 90/50

(52) U.S. Cl. CPC A61G 13/02; A61G 13/104; A61B 90/50; A61B 2090/508

(57) ABSTRACT

A modular multi-robotic manipulator system for multi-purpose image-guided surgical and diagnostic applications combining multiple open-chain and closed-chain robotic subsystems.

### CROSS-REFERENCES TO RELATED APPLICATIONS

This application claims the benefit of U.S. Provisional Application Ser. No. 62/722,488 filed on Aug. 24, 2018, and U.S. Provisional Application Ser. No. 62/722,506 filed on Aug. 24, 2018, and is a continuation-in-part application of U.S. application Ser. No. 17/271,030 filed on Feb. 24, 2021, the disclosures of which are hereby incorporated by reference in their entirety.

### FIELD OF INVENTION

This invention is directed to a modular multi-robotic delivery system for multi-purpose image-guided surgical and diagnostic applications.

### BACKGROUND

The subject invention is a modular multi-robotic manipulator system used for multi-purpose image-guided surgical and diagnostic applications. Unlike most medical robotic manipulators, which are designed to perform a specific task within a specific field of medicine, the subject invention's system's modular design allows for a wide range of unique configurations of its components making it suitable for almost all fields of medicine where robotic positioning can be used. Some notable applications include patient positioning for external beam radiotherapy (RT), stereotactic radiosurgery (SRS), and stereotactic body radiation therapy (SBRT); patient positioning combined with image-guided robotic implantation of radioactive sources in the field of brachytherapy; patient positioning combined with image-guided robotic surgical procedures (the particular embodiment used in the technical description); patient positioning for diagnostic imaging of any modality.

Most medical robotic manipulators are designed as either an open-chain or closed-chain system, each of which exhibits a particular set of distinct advantages and disadvantages. The selection of which type is used in the design is typically determined by analyzing the various tradeoffs and how they would impact the functionality within the specific field of application. In general, open-chain robotic manipulators provide a large working envelope, offering versatility in their range of motion, which is ideal for tasks requiring a high degree of freedom, such as surgical operations or patient rehabilitation. Open-chain systems also typically have simpler kinematics, which makes them easier to control and program. However, one of the main disadvantages of open-chain manipulators is that they tend to be less rigid and therefore less precise than their closed-chain counterparts, making them less suitable for tasks requiring high precision or the application of substantial forces.

On the other hand, closed-chain (or parallel) robotic manipulators are generally characterized by high stiffness, accuracy, and load capacity, making them advantageous for tasks that require precise control, such as microsurgery or specific types of tissue manipulation. The increased rigidity and accuracy of these systems are largely due to the fact that all of the links support the end-effector or tool. However, these advantages come with the cost of a more complex control system due to their intricate kinematics. Additionally, they typically have a smaller working envelope compared to open-chain systems, which could limit their utility in certain medical applications. Moreover, the increased stiffness of closed-chain systems can make them less compliant, which might be undesirable in scenarios where safety concerns mandate more compliant behavior, such as when direct physical interaction with a human operator or patient is involved.

Prior art robotic systems that were designed to perform surgery at multiple locations within the patient's body, using an open-chain design would allow the surgical tools to reach anywhere in the body, albeit at the cost of less precision and rigidity. Whereas, a closed-chain design would offer the necessary precision and rigidity but would be significantly limited in its range of motion.

The subject invention adopts a unique hybrid design that combines multiple open-chain and closed-chain robotic subsystems in a modular way, allowing it to perform different subtasks of the procedure that require a different set of advantages enabled by each type of robotic system. This combination allows the system to minimize the trade-offs that are present in medical robotic systems where only one type is used throughout.

### BRIEF DESCRIPTION OF THE DRAWINGS

FIG. 1 is a rear perspective view of the subject invention;

FIG. 2 is a sideview of the subject invention;

FIG. 3 is a front perspective view of the subject invention;

FIG. 4 is an alternate perspective view of an embodiment of the subject invention;

FIG. 5 is a perspective view of a component of the subject invention;

FIG. 6 is an exploded view of the component shown in FIG. 5;

FIG. 7 is a side view of a component of the subject invention;

FIG. 8 is a side view of a component of the subject invention;

FIG. 9 is a side view of a component of the subject invention;

FIG. 10 is a side view of a component of the subject invention;

FIG. 11a perspective view of a component of the subject invention;

FIG. 11b is an alternative embodiment of the component shown in FIG. 11a.

FIG. 12 is an exploded view of the component shown in FIG. 11a;

FIG. 13 a perspective view of a component shown in FIG. 12;

FIG. 14 an alternate perspective view of a component shown in FIG. 13;

FIG. 15 is a cross-sectional view of a component shown in FIG. 13;

FIG. 16 is a cross-sectional view of a component shown in FIG. 13;

FIG. 17 is a perspective view of an alternate embodiment of the subject application;

FIG. 18 is a side view of an alternate embodiment of the subject invention;

FIG. 19 is a side view of an alternate embodiment of the subject invention;

FIG. 20 is a perspective view of an alternate embodiment of the subject application;

FIG. 21 is side view of an alternate embodiment of the subject invention incorporating a hexapod robotic system;

FIG. 22 is a perspective view of the hexapod system shown in FIG. 21;

FIG. 23 is a perspective view of the embodiment shown in FIG. 18; and

FIG. 24 is a side of an view alternate embodiment of the subject invention.

### DETAILED DESCRIPTION OF THE PREFERRED EMBODIMENTS

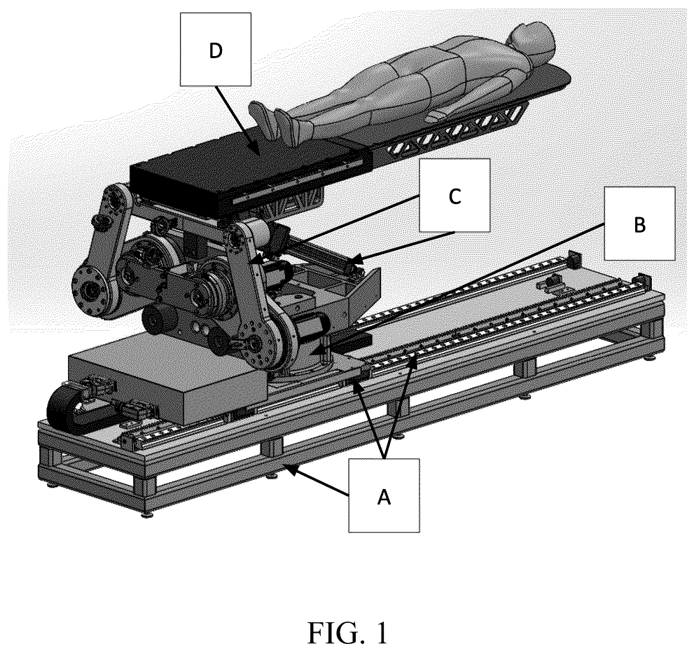

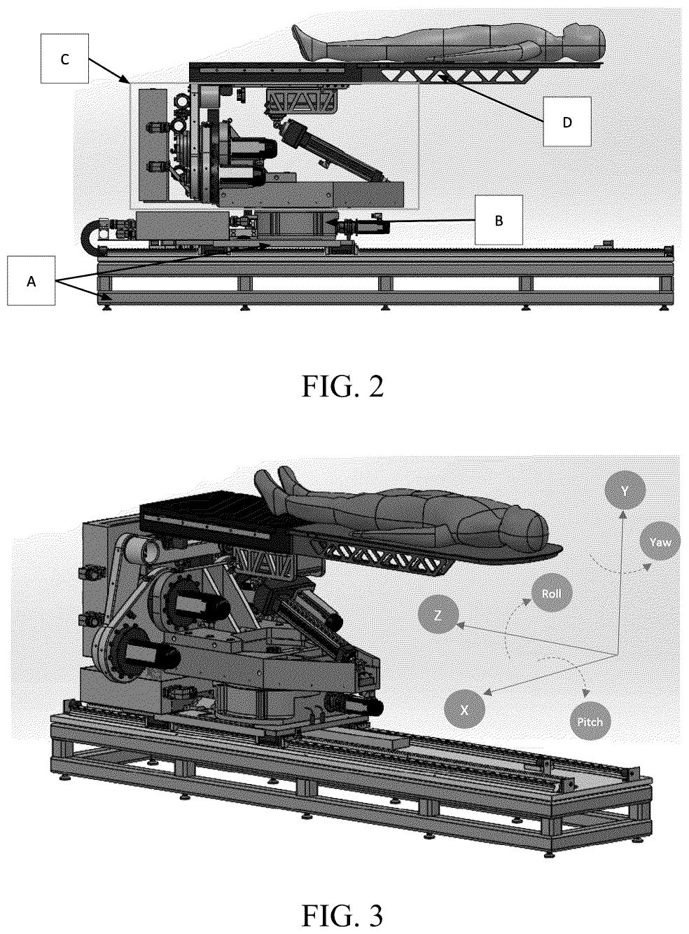

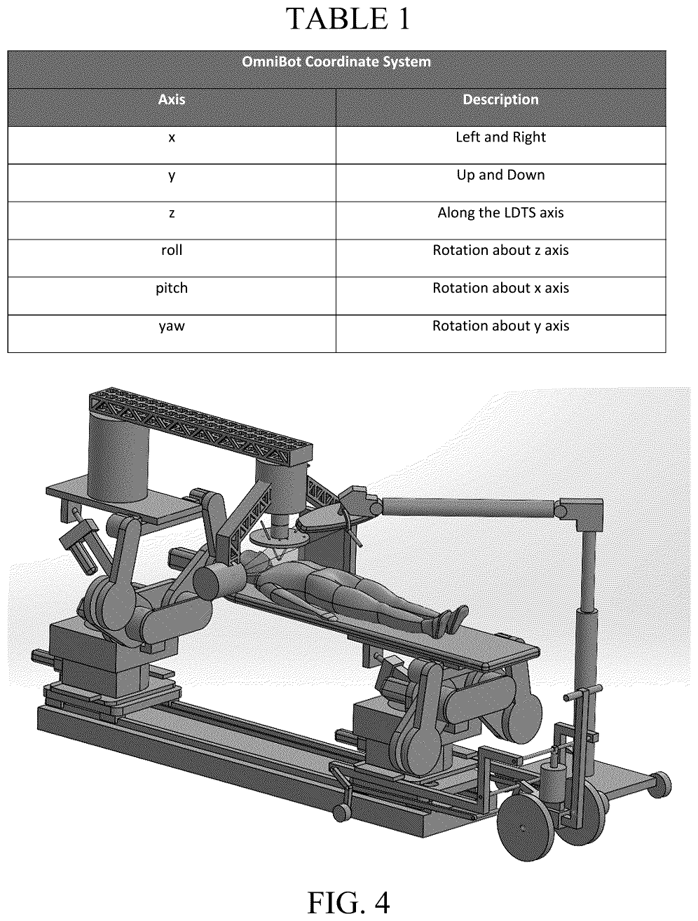

One embodiment of the subject invention (the System) is depicted in FIGS. 1 & 2. This embodiment will be referenced throughout the technical description of the system. Detailed descriptions of the possible variants that fall within the scope of the claims will be discussed in subsequent sections. As shown in FIGS. 1 and 2, the System consists of 4 major cooperating sub-systems:
A. The Linear Drive Train System (LDTS),
B. The Rotary Table System (RTS),
C. The Parallel Robotic Linkage System (PRLS), and
D. Various and Interchangeable End Effector Platforms (A carbon fiber table top for patient positioning is depicted in the current embodiment)

FIG. 3 and Table 1 show and describe the coordinate system for the System.

The subsequent subsections detail the technical design as well as the primary and secondary functions of each of the major components of the current embodiment of the System.

A. Linear Drive Train System:

The initial modular robotic subsystem within the overall System framework is the Linear Drive Train System (LDTS). The primary functions of the LTDS include:

Establishing a rigid connection between the System and the external environment,

Creating a fixed coordinate system (base frame) relative to the external world,

Providing structural support for the remainder of the System, and

Enabling 1-Degree of Freedom (DOF) for translational motion along the z-axis of the base frame, for one or more end-effector(s), with a long range of motion.

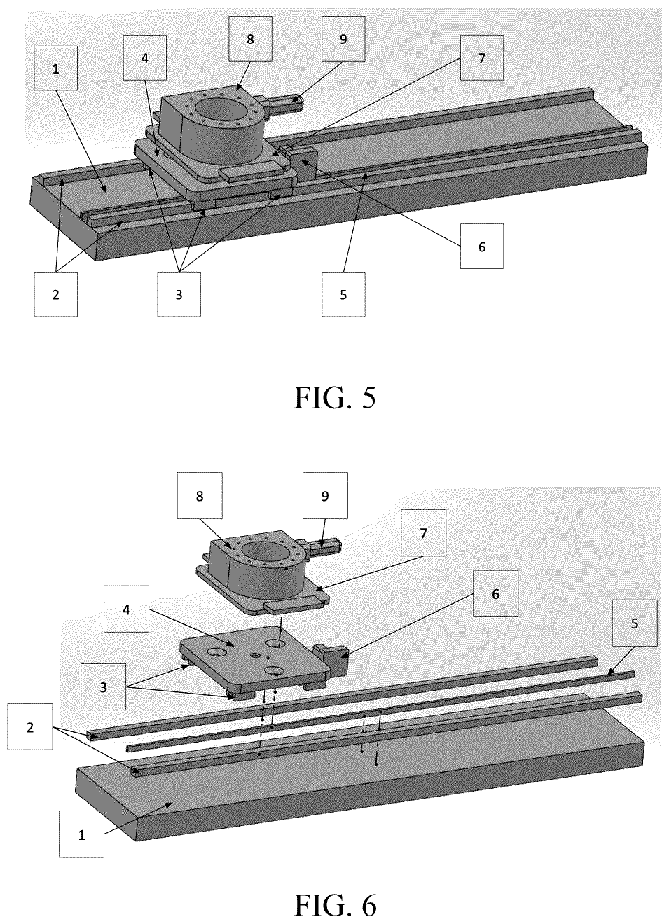

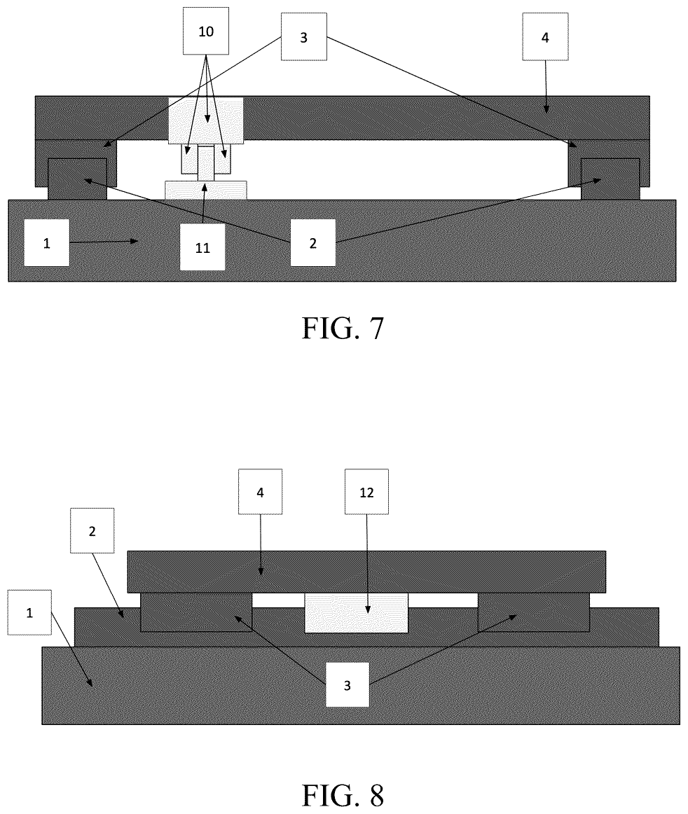

FIGS. 5 and 6 depict the LDTS and its major components.

The LDTS features a cuboidal structural body (1), capable of being firmly mounted to either a floor, a pit, walls, or even a ceiling in a clinical setting. The selected supporting structure must ensure sufficient structural support to the system, or be modified to do so. The orientation of the LDTS varies based on the specific application, with the current embodiment depicting a floor-mounted setup within a clinical setting.

Two parallel linear rail guide assemblies, comprising rails (2) and runner blocks (3)(3 of 4 visible in figure), are firmly affixed to the top surface of the structural body. Depending on the application, one or more linear platforms (4) are attached to four runner blocks (two per linear rail per platform). A servo-driven, backlash-free linear rack and pinion system drives the linear motion of these platforms. The linear rack (5) is firmly affixed to the structural body's top surface, while the servo-driven pinion(s) (6) are mounted on the linear platforms. This arrangement allows for the translation of one or more linear platforms along a singular rack.

Redundant encoding monitors the linear platforms' motion, with the primary encoder feedback sourced from the servo motor encoder on the drive side of the drivetrain. The secondary encoder feedback is sourced from a linear optical encoder system (not depicted in figures), with a stationary component firmly affixed to the structural body's top surface (1) and readheads attached to the linear platforms (4). This secondary feedback, being on the load side of the drivetrain, alerts the control system to any potential drivetrain failure.

The LDTS incorporates both primary and secondary brake systems. The primary brakes are servo motor brakes located on the drivetrain's drive side. As a secondary braking system, each linear platform is equipped with a spring-loaded, electromagnetic disc brake (10). Upon engagement, these brakes can clamp onto a linear bar which acts as the disk mounted on the structural body's (1) top surface, thereby operating as a load-side brake within the drivetrain. An alternative embodiment of the secondary brake system is a set of two linear rail guide brakes that act as additional runner blocks until engage, where they clamp onto the linear rails.

B. Rotary Table System

The second modular robotic component is the servo-driven Rotary Table System (RTS). Its primary functions include:
1. Providing 1-DOF rotational motion of the end effector about the y-axis of the base frame,
2. To physically link the Parallel Robotic Linkage System (PRLS) to the LDTS, and
3. To provide easy docking and undocking to and from the linear platform(s) of the LDTS.

The RTS features a cuboidal base plate (7) with two or more conical extrusions that extend below the bottom surface and onto which a servo-driven rotary table (8) is mounted to the top surface. The conical extrusions fit into the conical holes within the moving platform (4) of the LDTS and function primarily to precisely locate the RTS onto the moving platform. Conical extrusions and holes are used in the current embodiment to allow for self-alignment during the installation (docking) process, making it significantly easier while maintaining the original function; however, standard dowel pins and dowel holes could be used as an alternative solution, albeit with a more difficult docking process, since alignment would need to be done prior to lowering the RTS onto the moving platform. Once the top surface of the moving platform of the LDTS and the bottom surface of the base plate of the RTS are coincidentally aligned, the RTS can be secured to the moving platform by any standard means (nuts and bolts, screws, clamps, or latching mechanisms of any kind) given they do not uncouple during normal use of the OmniBot system.

On both sides of the base plate (7), a cuboidal flange is attached to the top surface and they extend outwards laterally from the base plate. These flanges allow the lifting arms of the Mobile Transportation System (MTS) to dock to and from the RTS for docking and undocking from the LDTS and for mobile transportation of the RTS—and the attached PRLS—to and from the work area (more details about this and its purposes will be discussed in subsequent sections).

The servo-driven rotary table enables bidirectional continuous rotation of its rotor with respect to its stator. The current embodiment features an open (hollow) center ring table that uses a barrel cam and cam follower system to drive the rotor with respect to the stator. The servo motor's output shaft is coupled to the input shaft of the rotary table by way of a cycloid gear inline reducer that reduces and increases the servo speed and torque respectively. The base frame of the PRLS is rigidly coupled to the rotor of the rotary table, such that when driven, the PRLS, and consequently the end effector's coordinate system, will rotate about the y-axis of the base frame coordinate system.

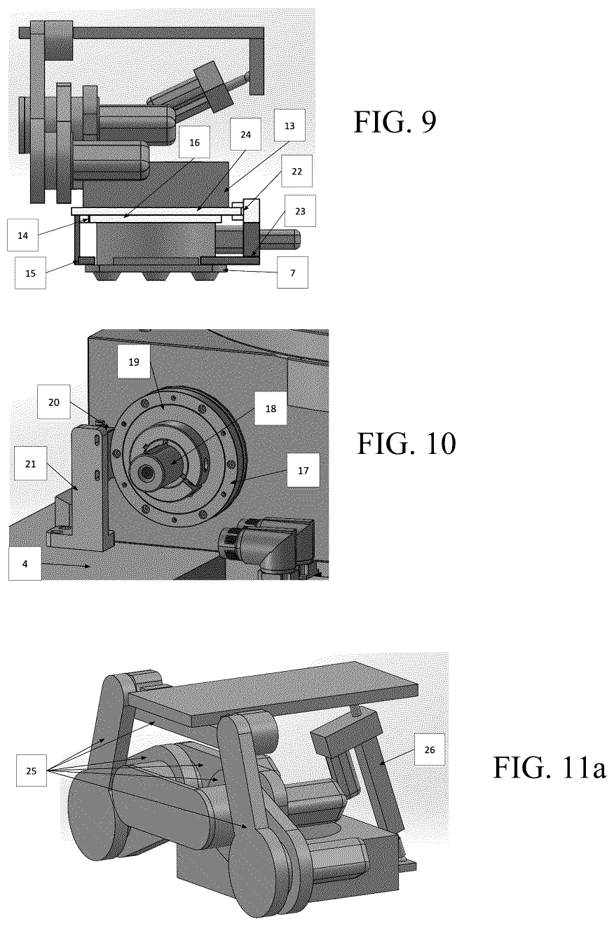

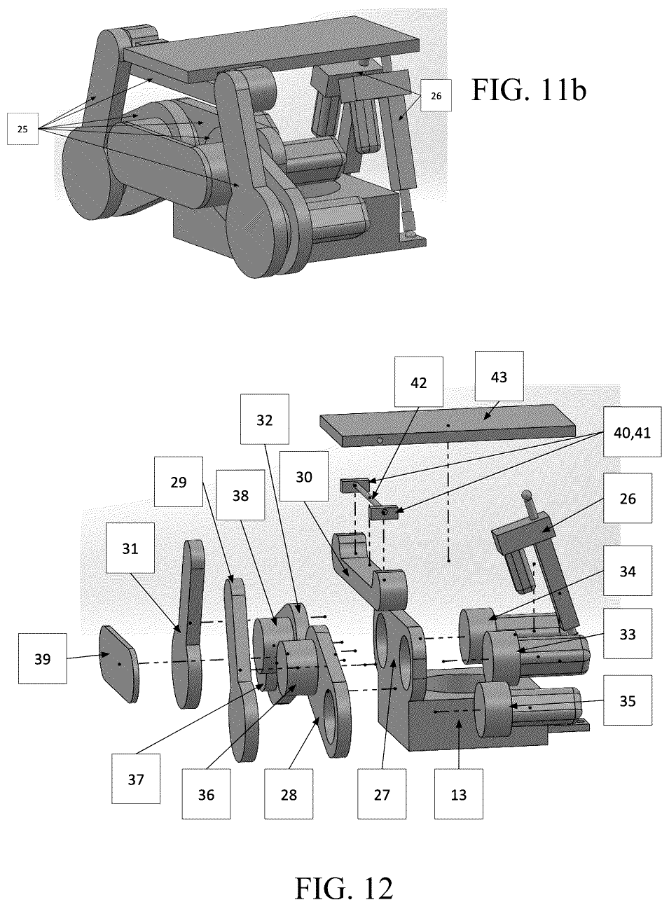

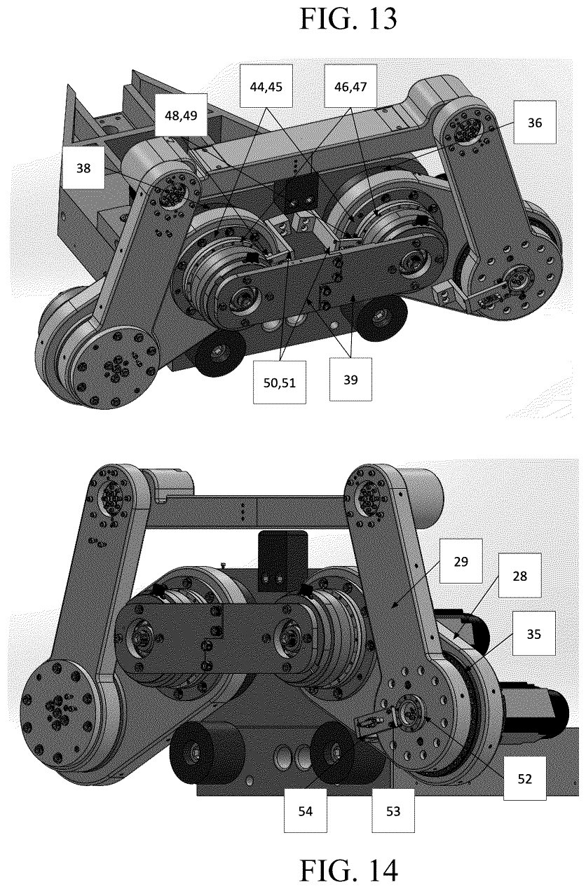

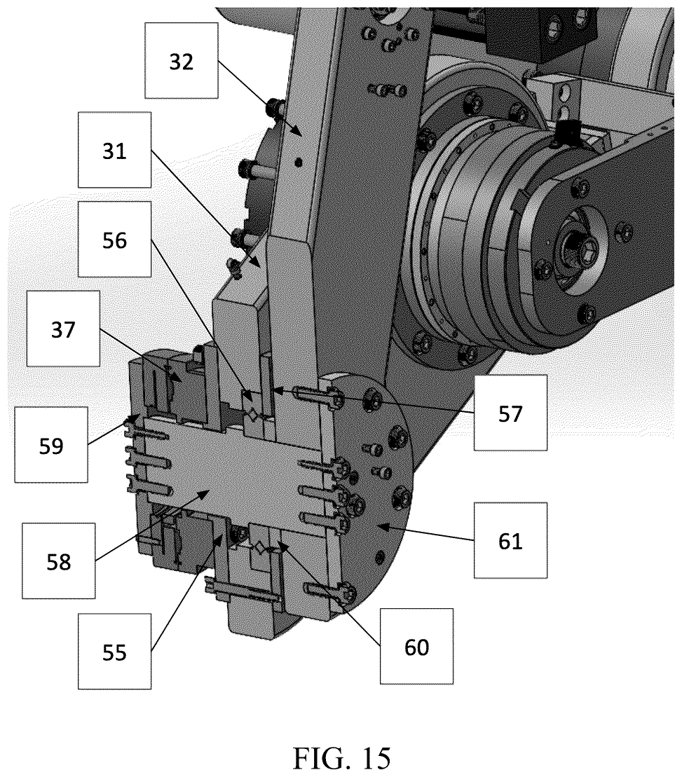

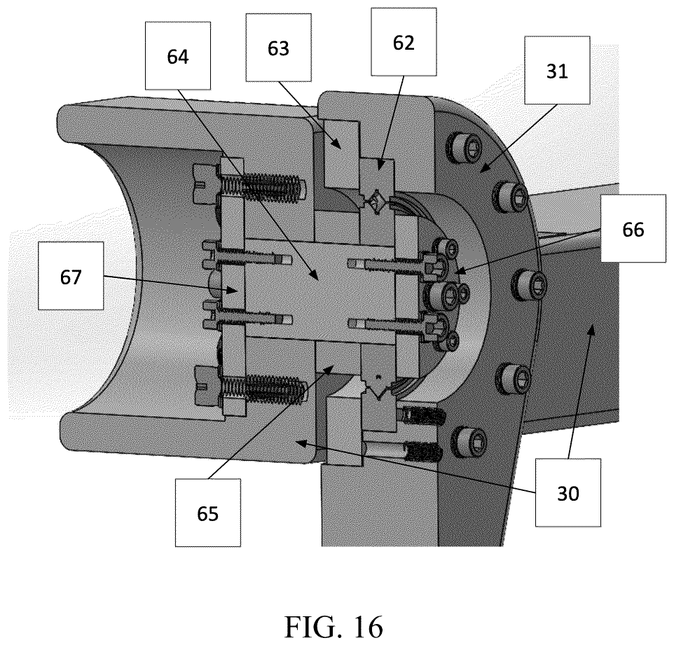

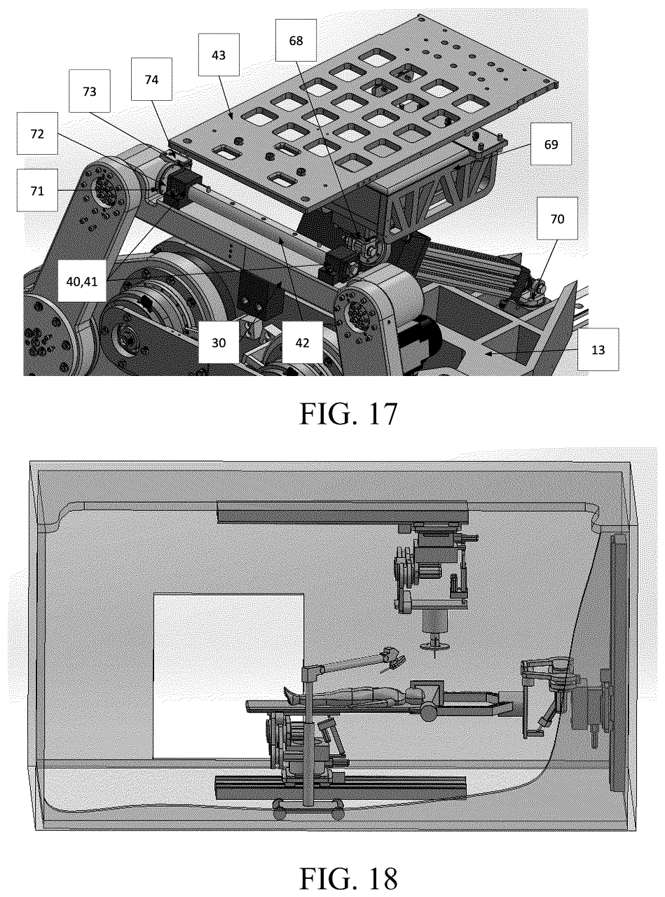

As with the LDTS, redundant encoding monitors the rotational motion about the y-axis, with the primary encoder feedback sourced from the servo motor encoder on the drive side of the drivetrain. The secondary encoder feedback is sourced from a rotary optical encoder system, with the encoder readhead (14) firmly affixed to the base plate of the RTS's (7) top surface by way of a bracket (15) and the optical encoder ring (16) firmly affixed to the bottom surface of the PRLS' base frame (13) (see FIG. 9). This secondary feedback, being on the load side of the drivetrain, provides the true orientation of the end-effector about the y-axis of the base frame coordinate system and it alerts the control system to any potential drivetrain failure. An alternate embodiment of the secondary encoder would have the optical encoder ring (17) mounted on the output shaft (18) of the rotary table by way of a cylindrical bracket (19), and the encoder readhead (20) mounted to the base plate of the RTS's (7) top surface by way of a bracket (21) (see FIG. 10). In this embodiment, the secondary encoder would still be able to alert the system if there is a drive-train failure due to the design of the rotary table, although the rotation of the rotor and secondary encoder would be subject to a gear ratio that the control system must account for.

Depending on the particular application of the subject system, the rotational range of motion can be limited by means of physical or software interlocks. In the current embodiment, software interlocks are used; however, one or more proximity switches, where the sensor(s) is(are) mounted on the stationary components of the RTS and the sensing element(s) is(are) mounted on the rotating component or vice versa, could also be used to limit the range of motion.

The RTS features primary and secondary braking systems. The primary brake is the servo motor brake located on the drivetrain's drive side. The secondary brake system consists of an electromagnetic disc brake (22) that is mounted to the top surface of the non-rotating base plate (7) of the RTS and a cylindrical disc that is mounted to the PRLS's base frame (13), just above the encoder ring (16). Upon engagement, the brake will clamp onto the disc halting the motion, and thereby operating as a load-side brake within the drivetrain.

As discussed above, the rotary table is hollow about its central rotational axis, which allows for electrical cables required by any component above it to be passed through uninhibited.

C. Parallel Robotic Linkage System

The third modular component of the System is the PRLS. Its primary functions include:
1. rigidly couples the end-effector and its coordinate system to the RTS,
2. provides 4-DOF motion to the end-effector about its own base coordinate system:
A. 2 translational DOF of the end effector along the x* and y axes w.r.t the PRLS' base coordinate system. Note, that this is different from the base frame coordinate system of the entire System as defined by the LTDS, except when the RTS is oriented such that the two are aligned. This and any other DOF that is defined w.r.t. a base coordinate system other than the global will have an asterisk attached.
B. 2 rotational DOF of the end effector about the z* and x* axes of the PRLS' base coordinate system.
3. to accommodate the end effector platform, onto which multiple medical tools can be equipped (e.g., a patient couch, an x-ray-based imaging system, surgical tools with various forms and functions, etc.).

The PRLS features a single-loop, 6-bar planar closed-chain linkage system (25), with 3 servo-driven and 3 passive revolute joints, uniquely coupled with one or more servo-driven linear actuators (26).

The stationary (in the PRLS base coordinate system) L-shaped base frame (13) is rigidly attached to the rotor of the rotary table. The vertical wall on the L-shaped base frame acts as the first (1 of 6 total) (27) linkage arms and is fixed with respect to the PRLS' base coordinate system. It features two large-bore through holes through which two servo-driven gearboxes (28 & 29) are passed through. The output shafts of the servo motors are directly coupled to the input shafts of the gearboxes. The gearbox stators are rigidly fixed to the outer surface of the L-shaped base frame's vertical wall. Rigidly attached to the output rotors of the gearboxes are the base-ends of linkage arms 2 (28) and 6 (32) (2 of the 3 driven linkage arms) and will rotate directly with the rotor when the gearbox is driven. On the outer surface and at the base end of linkages 2 and 6, a spring-loaded electromagnetic tooth brake (36 and 38) is rigidly attached by way of a custom Brake and Encoder Flange (44 & 45). The orientation of the brake is such that the rotor side is attached to the flange/linkage. A custom Brake Stator Bar (39) is attached to both of the stators and provides the necessary constraints to prevent either stator from rotating when the gearboxes are driven. An optical encoder ring (46 & 47) is rigidly attached to each of the Brake and Encoder Flanges and the encoder readheads (48 & 49) are rigidly mounted on the outer surface of the vertical wall by way of custom readhead flanges (50 & 51). When the brakes are released and the gearboxes are driven, the rotors, linkage arms (2 and 6), the optical encoders, and the brake rotors will all rotate directly with one another. In contrast, the readheads and brake stators remain stationary with respect to linkage arm 1.

At the top end of linkage arm 2 (i.e., the end closer to the end-effector), another servo-driven gearbox (35) is attached such that its stator is rigidly coupled to the outer wall of linkage arm 2 and passes through the large-bore through hole such that the servo motor is also on the inner side of the linkage arm. The base end of linkage arm 3 (29) (the third driven linkage arm) is rigidly attached to the stator of the gearbox directly with no Brake and Encoder Flange between them. The optical encoder ring (52) is attached directly to an extruded cylindrical section that extends from the outer surface of linkage arm 3 and its readhead (53) is rigidly coupled to linkage arm 2 by way of a custom readhead bracket (54). Similar to linkage arms 2 and 6, when driven by the gearbox rotor, linkage arm 3, and the optical encoder will rotate directly—while the readhead remains stationary—w.r.t linkage arm 2.

At the top end of linkage arm 6, the stator of another spring-loaded electromagnetic tooth brake (37) is rigidly attached to the inner surface by way of a custom brake flange (55) that exhibits a cylindrical hollow step inward that acts as an axial locating flange of the brake stator, ensuring that the brake is axially aligned with the semi-passive top end of linkage arm 6, and with the rotor oriented further inward. A ball-bearing (56) is press-fit within a counterbored section of the outer surface of linkage arm 3 such that its outer surface is coincident with the outer surface of linkage arm 6 and is held in place by a cylindrical flange (57) that is rigidly mounted on said surface of linkage arm 6. The bearing is also axially aligned with the tooth brake and semi-passive top end of the linkage arm 6. A custom shaft (58) extends through the bearing, linkage arm 6's semi-passive end, the tooth brake, and is rigidly mounted to a cylindrical flange (59) that is mounted to the outer surface of the tooth brake's rotor. At the other end of the shaft, a spacer block (60) and the semi-passive end of linkage arm 5 (31) is pressed over the shaft such that the outer surface of linkage arm 5 and the outer end of the shaft are coincident. A cylindrical flange (61) rigidly connects to the shaft and outer surface of linkage arm 5 and completes the semi-passive linkage joint between linkage arms 6 and 5. When the system is driven, the brake rotor, the shaft, the bearing rotor, and linkage arm 5 will rotate with respect to linkage arm 6.

At the top end of linkage arms 3 and 5 (depicted in FIG. 16) the outer race of a crossed roller bearing (62) is pressed into a cylindrical counter bore on the inner wall side. A hollow cylindrical plate (63) is placed within a larger counterbore and is rigidly screwed to the linkage arm with screws originating on the outer wall of the linkage arm, and this plate functions to prevent any axial motion of the outer race of the crossed roller bearing. A solid shaft (64) is pressed fit through the inner bore of the crossed roller bearing, an axial spacer (65), and the main through hole of the linkage arm 4 (32), such that its outer and inner surfaces are coincident with the outer surface of the crossed roller bearing's inner race and the inner surface of the counterbore in linkage arm 4. A solid cylindrical plate (66) is rigidly attached to the shaft and the outer surface of the inner race of the crossed roller bearing of linkage arms 3 and 5 and a another solid cylindrical plate (67) is rigidly connected to the other end of the shaft and linkage arm 4. The spacer thickness is designed to allow for the appropriate axial compression to reduce any unwanted play in the crossed roller bearing. Therefore, the revolute joints between linkage arms 3 and 4 and 3 and 5 are purely passive (i.e., they are not driven or braked), making linkage arm 4 a purely passive linkage arm.

This completes the single-loop, 6-bar planar closed-chain linkage system component of the PRLS. It exhibits 1 stationary linkage arm (1), 3 driven linkage arms (2, 3, and 6), one semi-passive linkage arm (5), and one purely passive linkage arm (4) in a closed chain. This 6-bar linkage system is redundantly encoded, with the primary encoder feedback sourced from the servo motors' encoders on the drive side of the drivetrain and the secondary encoder feedback sourced from the optical encoders on the load side of the drivetrain. It also exhibits primary and secondary brake systems. The primary brakes are servo motor brakes located on the drivetrain's drive side, and the secondary brakes are the spring-loaded electromagnetic tooth brakes located on the load side of the drivetrain. If 1 or more of the servo brakes fail, the load brakes will engage and stop the motion of the system. Furthermore, only 3 brakes are needed to prevent unwanted motion of the system, therefore 4 or more brakes would have to fail simultaneously or in succession for the system to collapse, making the system very safe. Those skilled in the art will understand that the servo-driven gearboxes and the load brakes could be located at any set of three joints throughout the closed chain, and in the current embodiment, the location of the gearboxes and brakes was chosen to optimize the spatial footprint and reduce the complexity of the cable management.

D. End Effector Platform

Two pillow block-bearing housing units (40 and 41) are rigidly attached on the left and right sides of the top surface of linkage arm 4 (i.e., 1 on the left and 1 on the right) and are axially aligned with the x-axis of the coordinate system of the PRLS. A quasi-cylindrical case-hardened shaft (42) is pressed within the inner bearing race on both bearing units and it is axially constrained geometrically. It is quasi-cylindrical because a flat surface is machined along its axis such that its normal vector is perpendicular to the rotational axis of the shaft, which allows for a flat plate (43) to be rigidly attached and aligned with screws and dowel pins respectively (more about this later). On either end of the shaft, an optical encoder ring (71) is rigidly coupled to the shaft by way of an encoder ring bracket (72), and its readhead (73) is rigidly mounted to linkage arm 4 via a readhead bracket (74). A cuboidal end-effector plate (43) is attached to the flat surface of the shaft and it always extends inward w.r.t the 6-bar linkage system, although it can also extend inward and outward depending on the particular application of the subject system. Towards the middle of the end-effector plate and mounted to its bottom side, a spherical rotational joint (68) is attached by way of a bracket (69) that orients it such that it faces another spherical rotational joint (70) which is rigidly mounted on the base of the PRLS at the most inward end (other variants are possible). A servo-driven linear actuator (26), which functions as a prismatic joint, is rigidly coupled to the spherical roller joints. In the current embodiment, the base joint of the linear actuator is attached to the end-effector platform's spherical rotational joint, and the rod end joint of the linear actuator is attached to the base frame; however, the overall functionality of the system remains unchanged if they were reversed. The current orientation is chosen strictly to optimize the range of motion and spatial footprint of the system. Furthermore, in the current embodiment 7 DOF are associated with the linear actuator (6 rotational from the 2 spherical roller joints and 1 prismatic joint), which allows the servo motor to maintain its orientation where the cable connectors are always oriented primarily downward, which minimizes the complexity of the cable management; however, the system will function with 6 DOF, albeit the orientation of the cable connectors will vary more with the configuration of the PRLS.

If the 6-bar linkage system is fixed and the linear actuator is driven, the end-effector platform will orbit the x-axis and therefore provide the 4th DOF of the PRLS. This motion is redundantly encoded, with the primary encoder feedback sourced from the servo motor's encoder on the drive side of the drivetrain and secondary (load) encoder feedback sourced from the rotational optical encoder on the rotational axis. Orbital motion about the x-axis can also be achieved by driving the 6-bar linkage system while either keeping the actuator fixed or driving it simultaneously. Additionally, to move the end-effector platform purely in x or y with no change in its orientation, both the 6-bar linkage system and the linear actuator must be driven in a coordinated manner as with any closed-chain robotic system. The PRLS can move the end-effector independently along or about the 4 DOF or any complex combination of one or more or all 4 DOF.

Various end-effector platforms that accommodate different clinical tools can be used, making the PRLS and the full subject system extremely versatile. Some notable embodiments with various end-effectors are depicted in the Figures below. The closed-chain PRLS is desired and chosen over an open-chain system as it allows for high-precision motion control, larger load capacity, and greater rigidity of the clinical tools used. It is decoupled from the translational motion along the z-axis and rotational motion about the y-axis to enable a larger range of motion about the patient and rotation away from the patient for other clinical activities (e.g., cleaning or replacing surgical tools) without losing the rigidity or precision control of the clinical tool that is almost always local.

As shown in FIG. 18, the subject invention allows the patient to be positioned beneath a surgical tool moveably mounted on a ceiling track and at the same time be connected to a diagnostic/imaging tool vertically moveably on a vertical surface mount.

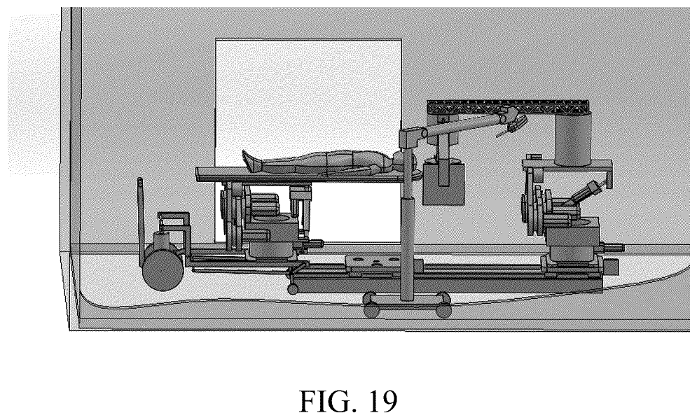

As shown in FIGS. 19 and 24, the subject invention includes a mobile wheel assembly apparatus that allows the positioning system to be moved from one location to another. This allows the patient to be moved from a diagnostic station or location to a surgical or treatment station in another room in the medical center.

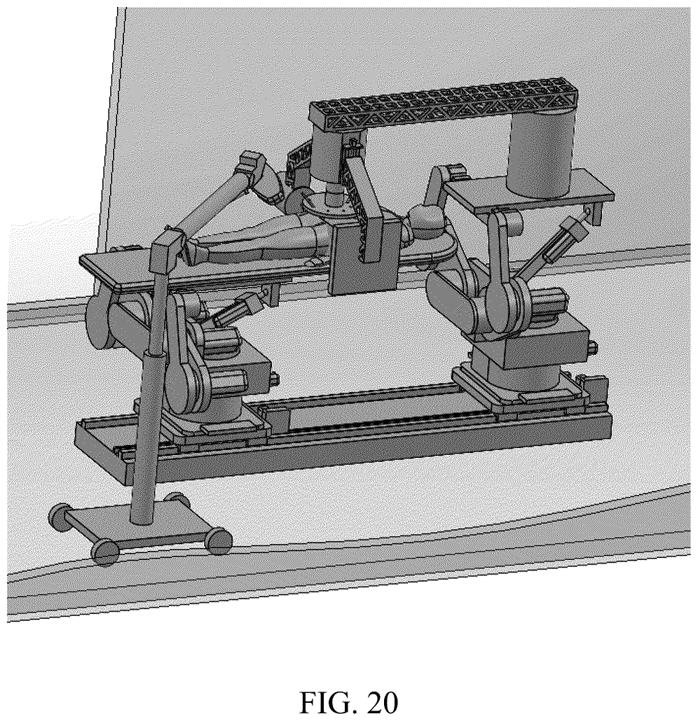

As shown in FIG. 20, the subject invention allows for two of the parallel robotic image systems to be mounted on the linear drive trail system thereby allowing movement and adjustment of the position of the patient and the medical tool.

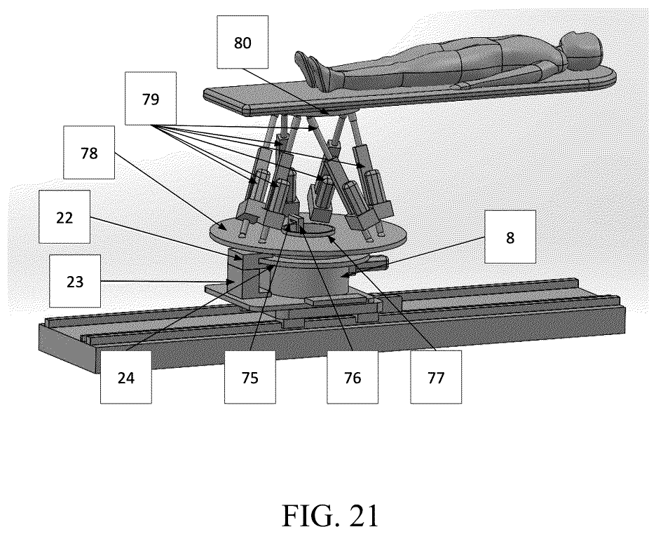

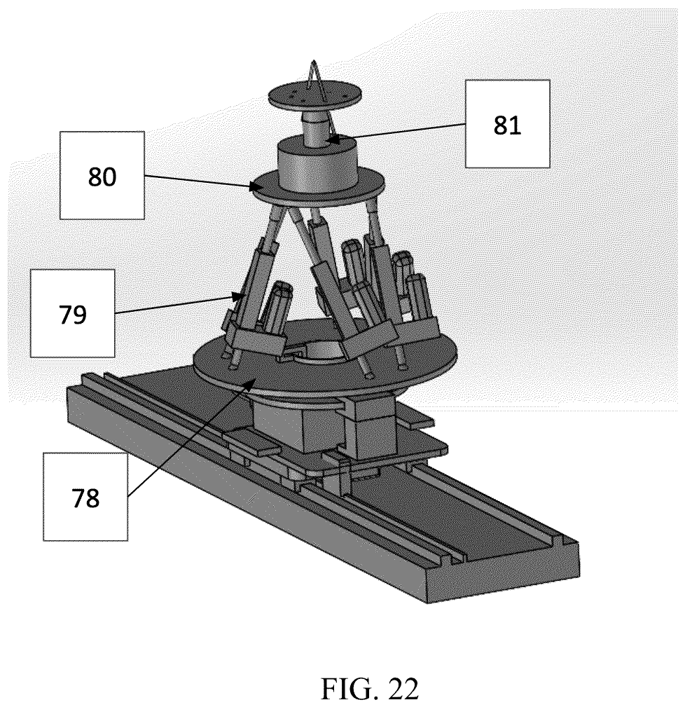

FIG. 21 depicts one variant of the System, wherein, the PRLS described previously can be replaced with another closed-chain robotic system. In the said embodiment, the closed-chain system is a 6-DOF hexapod, which consists of a base plate (78) that is mounted to the top of the cylindrical disc (24) of the rotary table's secondary brake system, and rotates directly with the rotary table, disc, and optical encoder ring (77). The base joints and platform joints of six linear prismatic servo-driven actuators (79) are mounted to the top surface of the base plate (78) and the bottom surface of the hexapod's platform plate (80) (another variant of the end-effector plate from the previous embodiment (43)) respectively. This figure also shows how the secondary encoder read-head bracket (76) can extend through the hollow rotary table and position the encoder read-head (75) such that it can read the encoder ring (77) mounted on the top of the hexapod's base plate. In this embodiment a patient positioning end-effector is shown; however, as with the preferred embodiment, various medical tools can be mounted to the end-effector platform, as depicted in FIG. 22, which shows a surgical tool 81 mounted on the hexapod's platform plate.

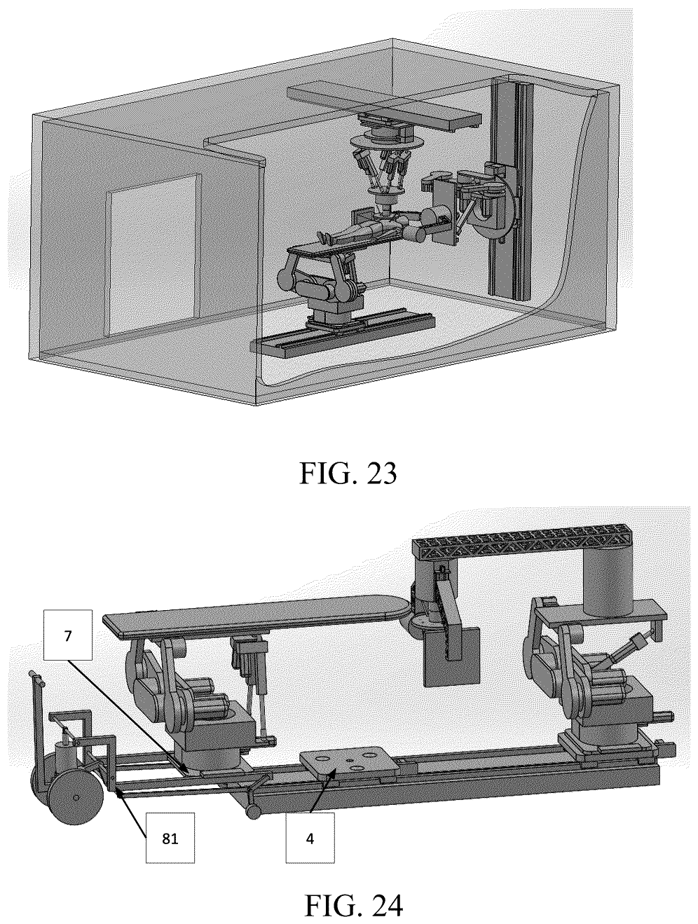

FIG. 23 depicts three embodiments of the System working together to perform image-guided robotic surgery. The three embodiments include:

E: a floor-mounted version featuring the PRLS and a patient positioning system attached. This version can precisely and rigidly position the patient during the surgical operation.

F: a wall-mounted version featuring the PRLS and a CBCT/X-ray diagnostic imaging system attached to the end-effector plate. This version can perform CBCT-based diagnostic imaging to accurately locate internal structures within the patient. It can also take X-ray images of the patient during treatment to ensure accurate positioning of the surgical tools within the patient.

G: a ceiling-mounted version featuring the hexapod-based embodiment with a surgical tool system (similar to the da Vinci Surgical System) attached. This version can precisely position the surgical tools robotically during the operation.

It is important to note that many other medical tools that require accurate positioning for diagnostic or therapeutic purposes can be utilized with the System, and the current list of embodiments is not exhaustive.

As shown in FIG. 24, another important feature of the System is that it is modular and easily transportable. The rotary table base plate (7) can be easily attached and detached from the LRS and transported by means of a custom transportation system (81) (which operates similarly to a pallet jack). Therefore, a hospital or clinic could have 2 or more LRS in different locations, and the RTS and PRLS can be attached to either depending on the current need of the System. This also allows for less downtime if maintenance on the RTS or PRLS, as they can be detached and transported to a maintenance area while another fully functional system is attached to the LRS and used while the other is repaired.

This figure also shows how two different components of the System (1 patient positioner and 1 surgical tool+x-ray imaging system can operate together on the same LRS).

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention. In addition, it should be understood that aspects of the various embodiments may be interchanged, both in whole, or in part. Therefore, the spirit, and scope of the invention should not be limited to the description of the preferred versions contained therein.

We claim:

1. A modular robotic patient and medical tool positioning system comprising:
a linear rail apparatus for supporting and positioning a medical tool in one axis;
said linear rail apparatus including two parallel rail guide assemblies;
a drive apparatus configured to move the medical tool along said linear rail apparatus;
primary and secondary brake systems provided on said linear rail apparatus;
a servo-driven rotary table system mounted on said rail apparatus and providing one degree of freedom continuous bi-directional rotation of the medical tool;
primary and secondary brake systems provided on the said rotary table apparatus;
a closed-chain servo-driven robotic manipulator mounted on the said rotary table and providing four (non-redundant degrees of freedom) or more (4 non-redundant plus n-additional redundant) degrees of freedom for the medical tool;
primary and secondary brake systems provided on the said closed-chain servo-driven robotic manipulator apparatus; and
a medical tool platform mounted on the said closed-chain servo-driven robotic manipulator to accommodate various medical tools.

2. The medical tool positioning system of claim 1 wherein:
the rotary table, closed-chain manipulator and medical tool platform can dock, undock, and re-dock onto another linear rail apparatus.

3. The medical tool positioning system of claim 1 wherein:
the various medical tools can be attached, detached, and exchanged.

4. The medical tool positioning system of claim 1 wherein:
the drive apparatus is a servo-driven linear rack and pinion system.

5. The medical tool positioning system of claim 1 wherein:
the drive apparatus is a servo-driven screw drive and spindle system.

6. The medical tool positioning system of claim 1 wherein:
the primary braking system is comprised of servo motor brakes positioned on the drivetrains drive side.

7. The medical tool positioning system of claim 1 wherein:
the secondary braking system is comprised of a spring-loaded electromagnetic disc brake.

8. The medical tool positioning system of claim 1 wherein:
the rotary table system includes primary and secondary braking system.

9. The medical tool positioning system of claim 8 wherein:
the primary braking system is a servo motor brake.

10. The medical tool positioning system of claim 8 wherein:
the secondary braking system is an electromagnetic disc brake.

11. The medical tool positioning system of claim 1 wherein:
the parallel robotic system includes a single-loop, 6-bar planar closed-chain
linkage system in operating agreement with one or more servo-driven linear actuators.

12. The medical tool positioning system of claim 1 wherein:
the parallel robotic system includes a six-degree-of-freedom servo-driven hexapod robotic system.

13. The medical tool positioning system of claim 1 wherein:
The linear rail system can be mounted to the floor, wall, or ceiling.

14. The medical tool positioning system of claim 1 further including:
a movable wheel assembly for removing the rotary table system from the linear rail apparatus.
